(19) United States
(12) Patent Application Publication
(10) Pub. No.: US 2008/0147070 A1
(43) Pub. Date: Jun. 19, 2008

(54) Reamer for surgical use

(76) Inventors: Hassler Michel, St Ismier (FR); Real Cecile, Grenoble (FR)

Correspondence Address: BACON & THOMAS, PLLC, 625 SLATERS LANE, FOURTH FLOOR, ALEXANDRIA, VA, 22314, US

(21) Appl. No.: 11/798,518

(22) Filed: May 15, 2007

(30) Foreign Application Priority Data
Dec. 13, 2006 (FR) 0655470

(51) Int. Cl. A61B 17/58

(52) U.S. Cl. 606/80; 606/79

(57) ABSTRACT

A cutting tool or reamer for surgical use comprising a shaft (10) and a rotary cutting element (23) driven by the shaft (10) is characterized in that the cutting element (23) is rotatable about an axis (21) that is substantially parallel to and offset from the axis (6) of the shaft (10).

The present invention concerns a cutting tool or reamer for surgical use.

The reamer according to the invention is specifically intended for carving beds in bony articular surfaces in order to accommodate implants or implant parts.

A known technique for treating a bony articular surface, prior to inserting an implant, consists of luxating the joint in order to expose the articular surface, then reaming this surface using a conventional surgical reamer. This technique is unsatisfactory because luxation damages the ligaments, the capsule and the cartilage of the joint. Moreover, for certain joints, such luxation is physically impossible or considered undesirable by surgeons because it is too invasive.

To avoid having to luxate a joint in order to prepare its articular surfaces, it is also known to use a reamer whose active cutting end is ball-shaped. The active end is inserted into the joint, between two bones, with its axis of rotation parallel to the surface to be treated. The surgeon sweeps the surface with the active end while the latter rotates until a bed of the desired shape is obtained. This method of reaming is tedious and makes it impossible, or at least very difficult, to obtain an evenly shaped bed.

Other techniques consist of resecting a large part of the bone opposite the articular surface to be carved or of distracting the joint in order to create enough space to insert a reamer at an angle. These techniques are very invasive and make it impossible, or at least difficult, to obtain beds of the right shape.

There are other known surgical reamers, used in the field of orthodontics, which comprise an angle gear that allows the active part of the reamer to form an angle with the main drive shaft. The space occupied by these reamers is such that using them to ream articular surfaces would also be very invasive.

The object of the present invention is to eliminate the aforementioned drawbacks and to propose a cutting tool for surgical use that facilitates the reaming of articular surfaces.

To this end, the subject of the invention is a cutting tool for surgical use comprising a shaft and a rotary cutting element driven by the shaft, characterized in that the cutting element is rotatable about an axis that is substantially parallel to and offset from the axis of the shaft.

As a result of this parallel offset between the cutting element and the drive shaft, the cutting element can be placed inside the joint with its axis of rotation perpendicular to the surface to be carved, without having to luxate the joint. The surface can thus be carved relatively easily and little invasively, and the bed obtained can have a very even shape.

The cutting tool according to the invention preferably also comprises a body comprising a tube inside which the shaft is guided in rotation and a wall located at one end of the tube and extending substantially perpendicular to the tube, the cutting element being mounted to said wall.

Advantageously, a handle extends substantially perpendicular to the tube.

The body can also comprise a skirt that extends from said wall, substantially parallel to the tube, so as to define with said wall a space in which elements for transmitting the rotation of the shaft to the cutting element are located. These transmission elements comprise, for example, a gear train.

The shaft is typically configured for being connected to a drive motor.

Advantageously, the rotary cutting element is snapped onto a spindle projecting from said wall of the body, and said wall comprises at least one through-hole for the insertion of a demounting tool for pushing the rotary cutting element in a direction that tends to unsnap it from said spindle projecting from said wall.

In another embodiment of the invention, the cutting tool also comprises a second cutting element rotatably mounted about an axis that is substantially parallel to and offset from both the axis of said rotary cutting element and the axis of the shaft, this second cutting element being driven by the rotation of said rotary cutting element and comprising teeth which are inscribed in a circle that overlaps a circle in which teeth of said rotary cutting element are inscribed.

Another subject of the present invention is a set comprising a cutting tool as defined above and a demounting tool comprising at least one pin that can be inserted into the through-hole of said wall of the cutting tool in order to push the rotary cutting element in a direction that tends to unsnap it from said spindle projecting from said wall.

Other features and advantages of the present invention will emerge from the reading of the following detailed description, given in reference to the attached drawings, in which:

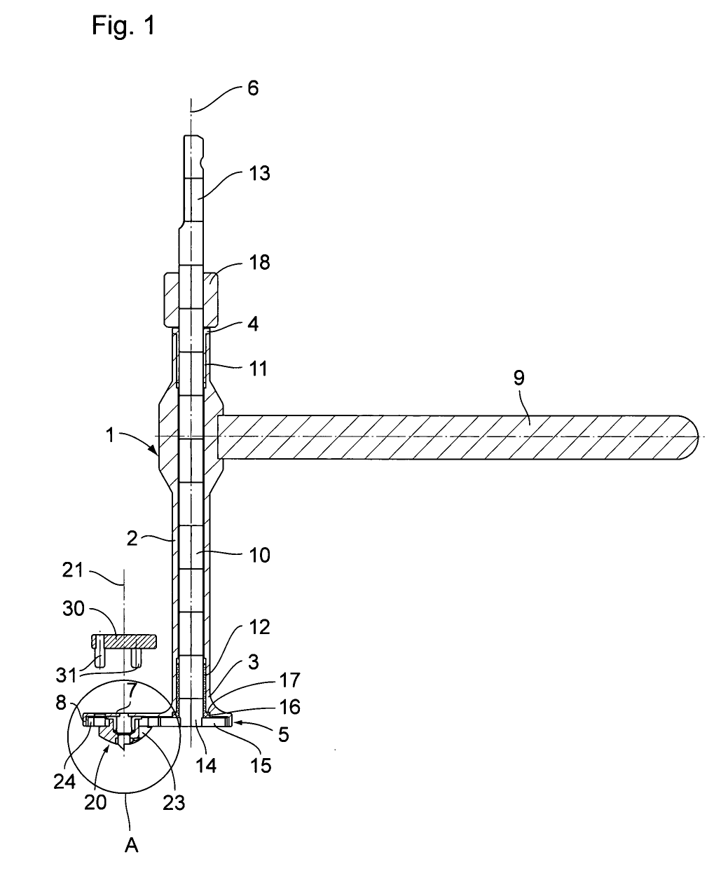

FIG. 1 is a front view in axial section of a reamer according to a first embodiment of the invention,

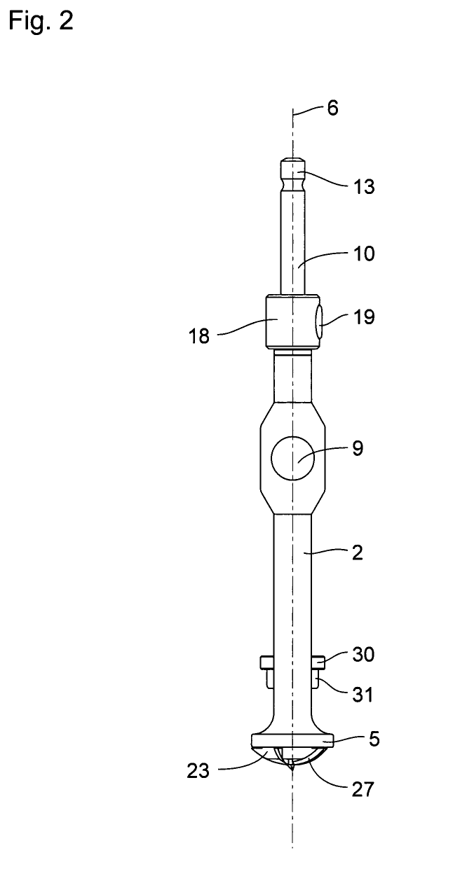

FIG. 2 is a side view of the reamer according to a first embodiment of the invention

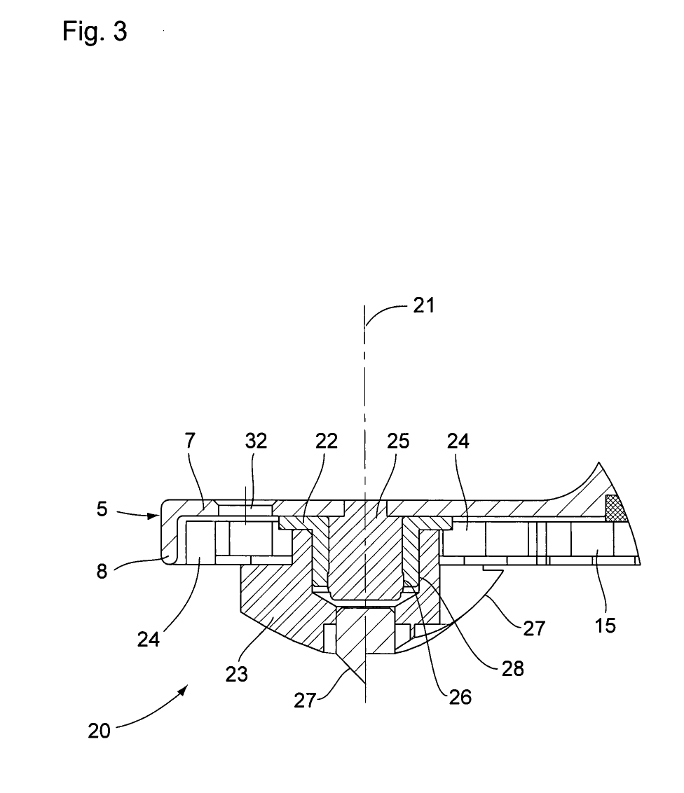

FIG. 3 is a detail in axial section of zone A in FIG. 1,

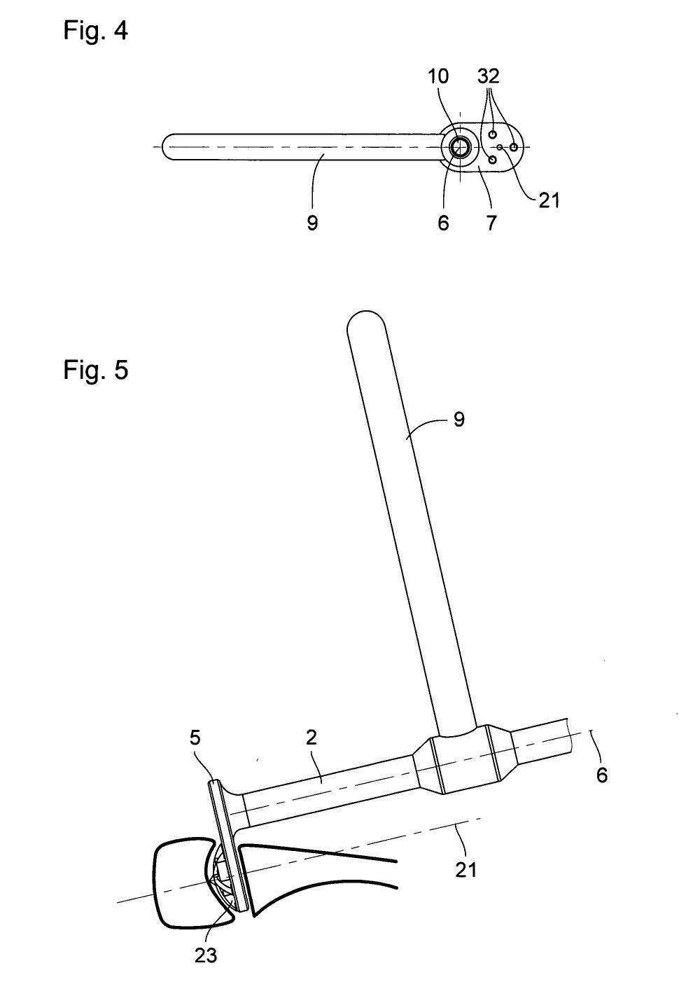

FIG. 4 is a top view of the reamer according to the first embodiment of the invention,

FIG. 5 is a schematic view showing the reamer according to the invention in the process of reaming an articular surface,

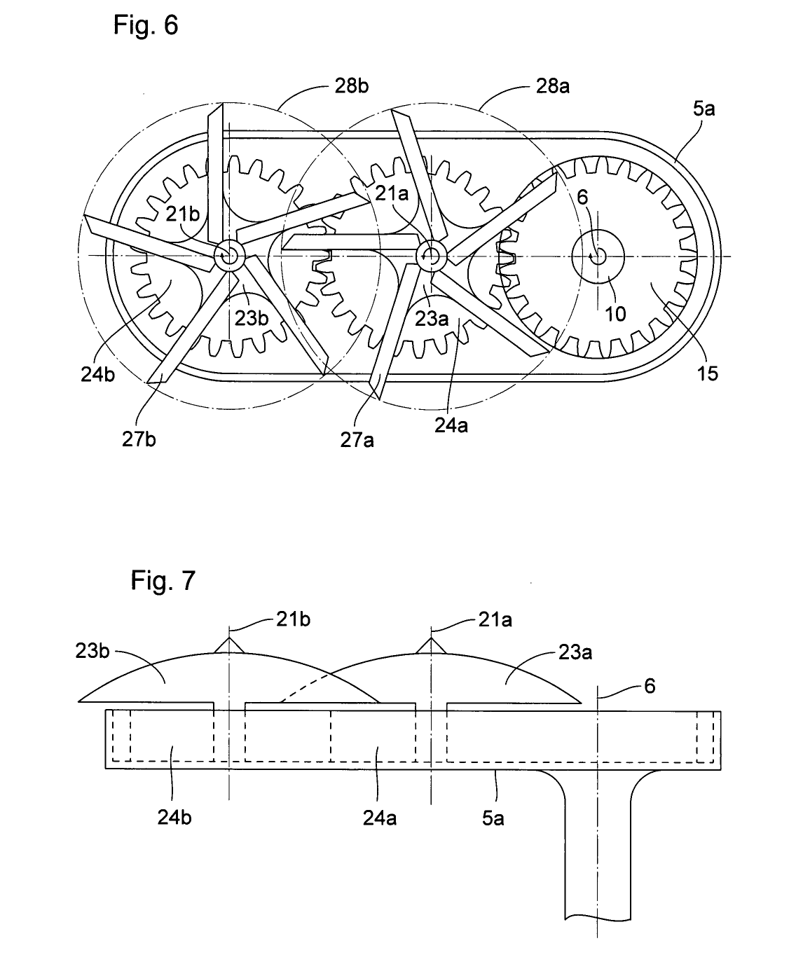

FIG. 6 is a schematic bottom view of the end of a reamer according to a second embodiment of the invention,

FIG. 7 is a schematic front view of the end of a reamer according to the second embodiment of the invention.

Referring to FIGS. 1 through 4, a reamer for surgical use according to the invention comprises a one-piece tool body 1 comprising a tube 2 that is open at both of its ends 3, 4 and a housing 5 whose shape is flat, oblong and off-center relative to the axis 6 of the tube 2, located in the extension of one 3 of the ends of the tube 2. The housing 5 is composed of an oblong, off-center wall 7 extending from the periphery of the tube 2 in a plane perpendicular to the axis 6, and a skirt 8 extending from the periphery of the wall 7, parallel to the axis 6, so as to axially terminate the body 1. A handle 9 is attached to the tube 2, perpendicular to the latter, by one of its ends.

A shaft 10 is inserted into the tube 2 coaxial to the latter, and is guided in rotation therein about the axis 6 by bearings 11, 12. The two ends 13, 14 of the shaft 10 are located outside the tube 2. The end 13 of the shaft 10 opposite from the housing 5 is configured for being connected to a drive motor (not represented). A toothed wheel 15 is mounted on the other end 14 of the shaft 10, coaxial with the shaft 10, inside the housing 5. The shaft 10 is axially locked inside the tube 2 in one direction by the wheel 15 resting against a widened end 16 of the bearing 12, which itself rests against an internal shoulder 17 of the tube 2, and in the other direction by a stop ring 18 surrounding the shaft 10 and resting against the end 4 of the tube 2, this stop ring 18 being rigidly connected to the shaft 10 by a locking screw 19.

Next to the wheel 15, inside the housing 5, is an element 20 rotatably mounted about an imaginary axis 21 that is parallel to the imaginary axis 6 of the tube 2 and the shaft 10 and is offset from this axis 6, i.e. not coaxial with the latter. The rotary element 20 comprises (cf. FIG. 3) a ring 22 made of plastic, for example polyether ether ketone (PEEK), a cutting element 23 constituting the active part of the reamer, and a toothed wheel 24. The cutting element 23 and the toothed wheel 24 both surround the ring 22 and are rigidly joined to the latter. The ring 22 is mounted on a cylindrical part 25, which constitutes a physical spindle that is symmetrical about the imaginary axis 21 and rigidly joined to the wall 7 of the housing 5 by one of its ends, the other end of the spindle 25 remaining free. The ring 22 is freely rotatable around the spindle 25 and is axially maintained on one side by the wall 7 and on the other side by a bulge 26 defined by the free end of the spindle 25. The toothed wheel 24 is coplanar with the toothed wheel 15 and meshes with the latter so that a rotation of the shaft 10 about its axis 6 under the action of the motor drives a rotation of the rotary element 20 about its axis 21. The cutting element 23 defines the end of the rotary element 20 and of the reamer in general, in the direction of the axes 6 and 21. The cutting element 23 has, for example, the general shape of a spherical cap, as shown, and includes cutting teeth 27 which, when the element 23 is driven in rotation, are capable of carving into a surface along the axis 21. The teeth 27 are located outside the housing 5. The toothed wheels 15, 24 themselves are inside the housing 5, protected by the skirt 8.

FIG. 5 shows how the reamer according to the invention can be used to ream an articular surface. The offset of the axis 21 from the axis 6 and the low overall height of the rotary element 20 and the wall 7 of the housing 5 allow the rotary element 20 to be easily inserted into a joint with its axis of rotation 21 perpendicular to the surface to be carved, the shaft 10 itself remaining outside the patient. In the example illustrated in FIG. 5, the rotary element 20 is inserted into a trapezometacarpal joint (thumb joint) in order to ream the articular surface of the trapezium so as to form a bed for the head of an implant, such as an implant of the type described in the present applicant's US patent application 2005/0119757.

With the reamer according to the invention, it is thus possible to ream an articular surface little invasively, in a joint that is neither luxated nor distracted, even in cases where the joint is small. In addition to hand surgery, the reamer according to the invention has advantageous applications, in particular, in the treatment of non-luxatable joints, such as the treatment of the vertebral articular surfaces (after a discectomy) or the shoulder joint.

Because the rotary element 20 and its cutting element 23 can be placed in front of the surface to be treated, with their axis of rotation 21 perpendicular to said surface, the reaming can be precise and a bed of very even shape can be obtained for the implant. Moreover, the transverse handle 9 increases the surgeon's comfort and the precision of his work by allowing him to hold the reamer with one hand on the handle 9 and the other hand on the handle (not represented) that contains the motor.

In a variant, the reamer according to the invention could be a manual tool that is not connectable to a motor. In that case, the end 13 of the shaft 10 would simply comprise a handle or a means for coupling with another manual tool.

Referring again to FIGS. 1 through 4, it may be seen that the distance between the axis 21 of the rotary element 20 and the axis 6 of the shaft 10 may be larger or smaller depending on the application. This distance will generally be greater than the sum of the respective maximum radii of the cutting element 23 and the shaft 10 so that the cutting element 23 and the shaft 10 are laterally spaced apart from one another, as in the example illustrated in the figures. In order to further distance the cutting element 23 from the shaft 10, it is possible to add one or more intermediate toothed wheels between the wheels 15, 24. Although they are identical in the example illustrated, the diameters of the toothed wheels 15, 24 and any intermediate toothed wheels could be different from one another.

It should be noted that other transmission means could be used in place of the gear train 15, 24 to transmit the rotation of the shaft 10 to the cutting element 23, such as coplanar grooved wheels rigidly joined to the shaft 10 and the cutting element 23, respectively, and connected to one another by an endless belt.

The rotary element 20 is simply snapped onto the spindle 25, owing to the elasticity of the ring 22, and can thus be easily detached from the body 1 in order to be cleaned or replaced. In its position illustrated in FIG. 3, the ring 22 is in a non-deformed, inactive position. The ring 22 is elastically compressed between the bulge 26 of the spindle 25 and a cylindrical wall 28 of the cutting element 23 in contact with the periphery of the ring 22 during the mounting of the rotary element 20 onto the spindle 25 and its demounting. To facilitate the demounting of the rotary element 20 from the spindle 25 and thus from the body 1, the present invention provides a tool 30 comprising pins 31 (cf. FIG. 1). These pins 31 can be inserted into through-holes 32 formed in the wall 7 of the housing 5 so as to come into contact with a surface of the rotary element 20, or more precisely, of the wheel 24 (cf. FIGS. 3, 4). Exerting pressure on the tool 30 then pushes the rotary element 20 axially toward the free end of the spindle 25 and forces the ring 22 to come unsnapped from the spindle 25. In the example illustrated (cf. FIGS. 1 and 4), there are three pins 31 and corresponding through-holes 32, distributed 120° apart from one another. There could, however, be a different number of them.

FIGS. 6 and 7 show a reamer for surgical use according to a second embodiment of the invention. The reamer according to this second embodiment differs from the one according to the first embodiment in that it comprises two cutting elements 23a, 23b rotatably mounted about respective imaginary axes 21a, 21b which are parallel to and offset from both one another and the axis 6 of the drive shaft 10. The cutting element 23a is identical to the cutting element 23 of the first embodiment except for its teeth 27a, which are longer, and is mounted to the housing 5a in the same way that the cutting element 23 is mounted to the housing 5. The cutting element 23b is mounted to the housing 5a in the same way as the cutting element 23a and is identical to the cutting element 23a except for its teeth 27b, which are reversed relative to the teeth 27a to allow for its direction of rotation, which is the reverse of the cutting element 23a. The cutting elements 23a, 23b are rigidly connected to and coaxial with toothed wheels 24a, 24b, which mesh with one another; the wheel 24a, which is located between the wheel 15 rigidly connected to the shaft 10 and the wheel 24b, also meshes with the wheel 15. The rotation of the shaft 10 is thus transmitted to the cutting element 23a by the gear train 15, 24a and to the cutting element 23b by the gear train 24a, 24b. The cutting elements 23a, 23b are also coplanar, and their teeth 27a, 27b are inscribed in respective overlapping circles 28a, 28b. The cutting elements 23a, 23b are nonetheless angularly offset from one another so that their respective teeth 27a, 27b do not meet.

The reamer according to this second embodiment makes it possible to produce an oblong hole by simultaneously forming two overlapping concave cavities. Depending on the application, it is clear that a higher number of cutting elements could be provided in order to obtain oblong holes of greater length.

1. Cutting tool for surgical use comprising a shaft and a rotary cutting element driven by the shaft, wherein the cutting element is rotatable about an axis that is substantially parallel to and offset from the axis of the shaft.

2. Cutting tool according to claim 1, including a body comprising a tube inside which the shaft is guided in rotation and a wall located at one end of the tube and extending substantially perpendicular to the tube, the cutting element being mounted to said wall.

3. Cutting tool according to claim 2, including a handle extending substantially perpendicular to the tube.

4. Cutting tool according to claim 2 wherein the body also comprises a skirt that extends from said wall, substantially parallel to the tube, so as to define with said wall a space in which elements for transmitting the rotation of the shaft to the cutting element are located.

5. Cutting tool according to claim 4, wherein said transmission elements comprise a gear train.

6. Cutting tool according to claim 1, wherein the rotary cutting element is driven by the shaft via a gear train.

7. Cutting tool according to claim 1, wherein the rotary cutting element is snapped onto a spindle projecting from a wall of a body 4 of the cutting tool.

8. Cutting tool according to claim 7, wherein said wall comprises at least one through-hole for the insertion of a demounting tool for pushing the rotary cutting element in a direction that tends to unsnap it from said spindle projecting from said wall.

9. Cutting tool according to claim 1, including a second cutting element rotatably mounted about an axis that is substantially parallel to and offset from both the axis of said rotary cutting element and the axis of the shaft, the second cutting element being driven by the rotation of said rotary cutting element and comprising teeth which are inscribed in a circle that overlaps a circle in which teeth of said rotary cutting element are inscribed.

10. Cutting tool according to claim 1, wherein the shaft is configured to be connected to a drive motor.

11. Set comprising a cutting tool according to claim 8, and a demounting tool comprising at least one pin configured to be inserted into the through-hole of said wall of the cutting tool in order to push the rotary cutting element in a direction that tends to unsnap it from said spindle projecting from said wall.
